(19) United States
(12) Patent Application Publication
(10) Pub. No.: US 2017/0253572 A1
(43) Pub. Date: Sep. 7, 2017

(54) USE OF AN ACIDIC SOLVENT AND WATER IN THE PRODUCTION OF 2,5-FURANDICARBOXYLIC ACID

(71) Applicant: BP Corporation North America Inc., Naperville, IL (US)

(72) Inventors: Victor A. Adamian, Naperville, IL (US); Joseph B. Binder, San Diego, CA (US); Ryan Shea, Naperville, IL (US)

(73) Assignee: BP Corporation North America Inc., Houston, TX (US)

(21) Appl. No.: 15/517,584

(22) PCT Filed: Oct. 7, 2015
(86) PCT No.: PCT/US2015/054501

§ 371 Date: Apr. 7, 2017

Related U.S. Application Data
(60) Provisional application No. 62/061,843, filed on Oct. 9, 2014.

(51) Int. Cl. C07D 307/68

(52) U.S. Cl. CPC C07D 307/68

(57) ABSTRACT

Methods for providing effective, efficient and convenient ways of producing 2,5-furandicarboxylic acid ate presented. In addition, compositions of 2,5-furandicarboxylic acid including 2,5-furandicarboxylic acid and at least one byproduct are presented. In some aspects, 4-deoxy-5-dehydroglucaric acid is dehydrated to obtain the 2,5-furandicarboxylic acid. A solvent, catalyst, and/or reactant may be combined with, the 4-deoxy-5-dehydroglucaric acid to produce a reaction product including the 2,5-furandicarboxylic acid. In some arrangements, the reaction product may additionally include water and/or byproducts.

### CROSS-REFERENCE

This application claims the benefit of U.S. provisional patent application Ser. No. 62/061,843 filed Oct. 9, 2014, and entitled “Use of an Acidic Solvent and Wafer in the Production of 2,5-Furandicarboxylic Acid” which is hereby incorporated herein by reference in its entirety.

### BACKGROUND

2,5-furandicarboxylic acid (FDCA) and FDCA esters are recognized as potential intermediates in numerous chemical fields. For instance, FDCA is identified as a prospective precursor in the production of plastics, fuel, polymer materials, pharmaceuticals, argicultural chemicals, and enhancers of comestibles, among others. Moreover, FDCAs are highlighted by the U.S. Department of Energy as a priority chemical for developing future “green” chemistry.

### SUMMARY

The following presents a simplified summary in order to provide a basic understanding of some aspects of the disclosure. The summary is not an extensive overview of the disclosure. It is neither intended to identify key or critical elements of the disclosure nor to delineate the scope of the disclosure. The following summary presents some concepts of the disclosure in a simplified form as a prelude to the description below.

Aspects of the disclosure provide effective, efficient, and convenient ways of producing 2.5-furandicarboxylic acid (FDCA). In particular, certain aspects of the disclosure provide techniques for dehydrating 4-deoxy-5-dehydroglucaric acid (DDG) to obtain FDCA. The dehydration reaction proceeds by combining one or more catalysts and/or one or more solvents with a DDG starting material. In some instances, the catalyst may act as a dehydrating agent and may interact with hydroxyl groups on the DDG thereby encouraging elimination reactions to form FDCA. The catalyst and/or solvents may assist the dehydration reaction thereby producing increased yields of FDCA.

In a first embodiment, a method of producing FDCA includes bringing DDG into contact with a solvent in the presence of a catalyst (e.g., combining DDG, a solvent, and a catalyst in a reactor), wherein the catalyst is selected from the group consisting of a bromide salt, a hydrobromic acid, elemental bromine, and combinations thereof, and allowing DDG to react to produce FDCA, any byproducts, and water.

In other embodiments, a method of producing FDCA includes bringing DDG into contact with a solvent in the presence of a catalyst (e.g., combining DDG, a solvent, and a catalyst in a reactor), wherein the catalyst is selected from the group consisting of a halide salt, a hydrohalic acid, elemental ion, and combinations thereof, and allowing DDG to react to produce FDCA, any byproducts, and water.

In another embodiment, a method of producing FDCA includes bringing DDG into contact with an acidic solvent in the presence of water, and allowing DDG, the acidic solvent, and water to react with each other to produce FDCA, any byproducts, and water.

In some embodiments, a method of producing FDCA includes bringing DDG into contact with a carboxylic acid, and allowing DDG and the carboxylic acid to react with each other to produce FDCA, any byproducts, and water.

These features, along with, many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIG. 1 illustrates a graph that depicts the benefit of using water with an acidic solvent according to one or more embodiments.

### DETAILED DESCRIPTION

Various examples, aspects, and embodiments of the subject matter disclosed here are possible and will be apparent to the person of ordinary skill in the art, given the benefit of this disclosure. In this disclosure reference to “certain exemplary embodiments” or aspects (and similar phrases) means that those embodiment or aspects are merely non-limiting examples of the subject matter and that there likely are other alternative embodiments or aspects which are not excluded. Unless otherwise indicated or unless otherwise clear from the context in which it is described, alternative elements or features in the embodiments and examples below and in the Summary above are interchangeable with each other. An element described in one example may be interchanged or substituted for one or more corresponding elements described in another example. Similarly, optional or non-essential features disclosed in connection with a particular embodiment or example should be understood to be disclosed for use in any other embodiment of the disclosed subject matter. More generally, the elements of the examples should be understood to be disclosed generally for use with other aspects and examples of the products and methods disclosed herein. A reference to a component or ingredient being operative, i.e., able to perform one or move functions, tasks and/or operations or the like, is intended to mean that it can perform the expressly recited function(s), task(s) and/or operation(s) in at least certain embodiments, and may well be operative to perform also one or more other functions, tasks and/or operations.

While this disclosure includes specific examples, including, presently preferred modes or embodiments, those skilled in the art will appreciate that there are numerous variations and modifications within the spirit and scope of the invention as set forth in the appended claims. Each word and phrase used in the claims is intended to include all its dictionary meanings consistent with its usage in this disclosure and/or with its technical and industry usage in any relevant technology area. Indefinite articles, such as “a,” and “an” and the definite article “the” and other such words and phrases are used in the claims in the usual and traditional way in patents, to mean “at least one” or “one or more.” The word “comprising” is used in the claims to have its traditional open-ended meaning, that is, to mean that the product or process defined by the claim may optionally also have additional features, elements, steps, etc. beyond those expressly recited.

### Dehydration Reaction of DDG to FDCA

The present invention is directed to synthesizing 2,5-disubstituted furans (which may include, e.g., FDCA) by the dehydration of oxidized sugar products (which may include, e.g., DDG). In accordance with some aspects of the invention, the dehydration methods produce higher yields and/or higher purity 2,5-disubstituted furans than previously known dehydration reactions.

In certain aspects, the DDG may be a DDG salt and/or a DDG ester. For example, esters of DDG may include dibutyl ester (DDG-DBE). Salts of DDG may include DDG 2K, which is a DDG dipotassium salt. The FDCA may be an FDCA ester (e.g., FDCA-DBE). For example, a starting material of DDG-DBE may be dehydrated to produce FDCA-DBE. For ease of discussion, “DDG” and “FDCA” as used herein refer to DDG and FDCA genetically (including but not limited to esters thereof), and not to any specific chemical form of DDG and FDCA. Specific chemical forms, such as esters of FDCA and DDG, are identified specifically.

DDG is dehydrated to produce FDCA. The dehydration reaction may additionally produce various byproducts in addition to the FDCA. In some aspects, DDG is combined with a solvent (e.g., an acidic solvent) and/or a catalyst, and allowed to react to produce FDCA. DDG may be dissolved in a first solvent prior to adding the DDG to a catalyst. In some aspects, DDG may be dissolved in a first solvent prior to adding the DDG (i.e., the dissolved DDG and the first solvent) to a catalyst and/or a second solvent. In certain aspects, DDG is dissolved in water prior to adding the DDG to a catalyst and/or an acidic solvent. It is generally understood that by dissolving the DDG in water prior to adding any other component (e.g., a catalyst) causes a more efficient reaction from FDCA to DDG. A few reasons for why a more efficient reaction may occur include, by dissolving DDG-2K in water prior to adding a catalyst or acidic solvent, the DDG-2K Is more effective in solution; DDG may adopt its preferred form when first dissolved in water; and DDG in solution may increase yields of FDCA.

In certain aspects, the catalyst is a solvent. In some aspects, the catalyst also acts as a dehydrating agent. The catalyst may be a salt, gas, elemental ion, and/or an acid. In certain aspects, the catalyst and/or solvent is selected from one or more of an elemental halogen (e.g., elemental bromine, elemental chlorine, elemental fluorine, elemental iodine, and the like), hydrohalic acid (e.g., hydrobromic acid, hydrochloric acid, hydrofluoric acid, hydroiodic acid, and the like), alkali and alkaline earth metal salts (e.g., sodium bromide, potassium bromide, lithium bromide, rubidium bromide, cesium bromide, magnesium bromide, calcium bromide, strontium bromide, barium bromide, sodium chloride, potassium chloride, lithium chloride, rubidium chloride, cesium chloride, magnesium chloride, calcium chloride, strontium chloride, barium chloride, sodium fluoride, potassium fluoride, lithium fluoride, rubidium fluoride, cesium fluoride, magnesium fluoride, calcium fluoride, strontium fluoride, barium fluoride, sodium iodide, potassium iodide, lithium iodide, rubidium iodide, cesium iodide, magnesium iodide, calcium iodide, strontium iodide, barium iodide, other alkali or alkaline earth, metal salts, other salts in which at least some of the negative ions are halides, and the like), acetyl chloride, other acid halides or activated species, other heterogeneous acid catalysts, trifluoroacetic acid, acetic acid, water, methanol, ethanol, 1-propanol, 2-propanol, 1-butanol, n-methylpyrrolidone acid, propionic acid, butyric acid, formic acid, other ionic liquids, nitric acid, sulfuric acid, phosphoric acid, methanesulfonic acid, p-toluenesulfonic acid, other supported sulfonic acids (e.g., nation, Amberlyst®-15, other sulfonic acid resins, and the like), heteropoly acids (e.g., tungstosilicic acid, phosphomolybdic acids phosphotungstic acid, and the like), acids with a first pKa less than 2, and other supported organic, or inorganic acids, and supported or solid acids. A catalyst may be obtained from any source that produces that catalyst in a reaction mixture (e.g., bromine containing catalyst may be obtained from any compound that produces bromide ions in the reaction mixture).

Acetic acid is a particularly desirable solvent as the ultimate FDCA product has a lower color value, e.g. it is whiter than products produced with other solvents. Trifluoroacetic acid and water are additional preferred solvents for the production of FDCA. Additionally, the combinations of trifluoroacetic acid with water and acetic acid with water are particularly desirable for being low cost solvents.

It is generally understood that the dehydration of DDG to FDCA by the methods discussed herein provide molar yields of FDCA larger than those obtained from previously known dehydration reactions. In some aspects, the dehydration reaction yields at least 20%, at least 30%, at least 40%, at least 50%, at least 55%, at least 60%, at least 65%, at least 70%, at least 75%, at least 80%, at least 85%, at least 90%, at least 95%, or at. least 99% molar yield of FDCA that may be produced from DDG as the starting material. In other aspects, the dehydration reaction yields between 20% and 100%, between 20% and 90%, between 20% and 80%, between 30% and 100%, between 30% and 90%, between 30% and 80%, between 40% and 100%, between 40% and 90%, between 40% and 80%, between 40% and 70%, between 40% and 60%, between 50% and 100%, between 50% and 90%, between 50% and 80%, between 50% and 70%, between 55% and 95%, between 55% and 90%, between 55% and 85%, between 55% and 80%, between 55% and 75%, between, 55% and 70%, between 60% and 99%, between 60% and 95%, between 60% and 90%, between 60% and 85%, between 60% and 80%, between 65% and 99%, between 65% and 95%, between 65% and 90%, between 65% and 85%, between 65% and 80%, between 70% and 99%, between 70% and 95%, between 70% and 90%, between 70% and 85%, between, 75% and 99%, between 75% and 95%, between 75% and 90%, between 75% and 85%, between 80% and 99%, between 80% and 95%, between 85% and 99%, or between 90% and 99% molar yield of FDCA that may be produced from DDG as the starling material.

The FDCA produced via the dehydration reaction may be isolated and/or purified. Suitable isolation or purification techniques include filtrating and washing the FDCA product with water or recrystallizing the FDCA from water.

The purified FDCA may have multiple uses in the industry such as an alternative to terephthalic acid in producing polyethylene terephthalate (PET). PET is commonly used to manufacture polyester fabrics, bottles, and other packaging. FDCA may also be a precursor for adipic acid, jet fuels, other diols, diamine, or dialdehyde based chemicals.

In one aspect, the process described above is conducted by adding DDG and a catalyst and/or a solvent into a reaction vessel provided with a stirring mechanism and then stirring the resulting mixture. The reaction vessel may be a batch or a continuous reactor. A continuous reactor may be a plug flow reactor, continuous stirred tank reactor, and a continuous stirred tank reactor in series. In some aspects, the reaction vessel may be selected for a dehydration reaction based on its metallurgy (e.g., a zirconium reactor may be selected over a teflon reactor for reactions utilizing bromine). A reaction vessel may be a zirconium reactor, a teflon reactor, a glass-lined reactor, or the like. The temperature and pressure within the reaction vessel may be adjusted as appropriate. The DDG may be dissolved in water or another solvent prior to adding the DDG (i.e., the dissolved DDG and solvent) to the reaction vessel. In certain aspects, DDG is mixed with the solvent at a temperature in the range of 5° C. to 40° C., and in more specific aspects at about 25° C., to ensure dissolution in the solvent before the catalyst is added and reaction is initiated. Additionally and/or alternatively, the catalyst may be mixed with the solvent at room temperature to ensure dissolution in the solvent before being added to the DDG.

In some aspects, the process includes removing water produced during the reaction. Reducing at least some of the water produced may reduce or eliminate side reactions and reactivate the catalysts. As a consequence higher product yields may be obtained. Any suitable means may be used to regulate the amount of water in the reaction vessel such as use of a water content regulator.

The manufacturing process of FDCA may be conducted in a batch, a semi-continuous, or a continuous mode. In certain aspects, the manufacture of FDCA operates in a batch mode with increasing temperatures at predefined times, increasing pressures at predefined times, and variations of the catalyst composition during the reaction. For example, variation of the catalyst composition during reaction can be accomplished by the addition of one or more catalysts at predefined times.

The temperature and pressure typically can be selected from a wide range. However, when the reaction is conducted in the presence of a solvent, the reaction temperature and pressure may not be independent. For example, the pressure of a reaction mixture may be determined by the solvent pressure at a certain temperature. In some aspects, the pressure of the reaction mixture is selected such that the solvent in mainly in the liquid phase.

The temperature of the reaction mixture may be within the range of 0° C. to 180° C. and in certain aspects may be within tire range of 20° C. to 100° C., and in more specific aspects within the range of 60° C. to 100° C. A temperature above 180° C. may lead to decarboxylation to other degradation products and thus such higher temperatures may need to be avoided.

In some aspects, a dehydration reaction may run for up to 48 hours. In alternative aspects, a dehydration reaction may run for less than 5 minutes (i.e., the dehydration reaction is at least 95% complete within 5 minutes). In certain preferred examples, a dehydration reaction may occur within the time range of 1 minute to 4 hours. (i.e., the dehydration reaction of the reaction mixture is at least 95% complete within 1 minute to 4 hours). In some aspects the reaction of the reaction mixture is at least 95% complete within no more than 1 minute, 5 minutes, 4 hours, 8 hours or 24 hours. The length of the reaction process may be dependent on the temperature of the reaction mixture, the concentration of DDG, the concentration of the catalyst, and the concentration of other reagents. For example, at low temperatures (e.g., at or near the freezing point of the selected solvent) the reaction may run for up to two days, but at high temperatures (e.g., above 100° C.) the reaction may run for less than five minutes to achieve at least 95% completion.

Upon completion of the reaction process, a reaction product may be formed including FDCA and various byproducts. The term “byproducts” as used herein includes all substances other than 2,5-furandicarboxylic acid and water. In some aspects, the number, amount, and type of byproducts obtained in the reaction products may be different than those produced using other dehydration processes. Undesirable byproducts, such as 2-furoic acid and lactones, may be produced in limited amounts. For example, byproducts may include,

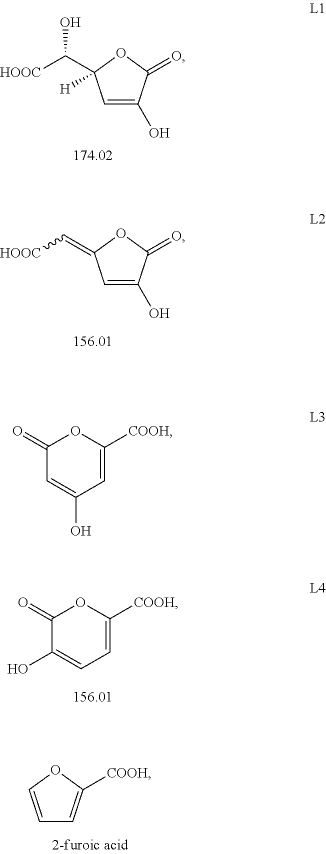 [SMILES: O=C1O[C@H]([C@@H](O)C(=O)O)C=C1O ; O=C(O)C=C1C=C(O)C(=O)O1 ; O=C(O)c1cc(O)cc(=O)o1 ; O=C(O)c1ccc(O)c(=O)o1 ; O=C(O)c1ccco1]

and the like. In certain aspects, undesirable byproducts may also include DDG-derived organic compounds containing at least one bromine atom. A reaction product may contain less than 15%, alternatively less than 12%, alternatively 10% to 12%, or preferably less than 10% byproducts. The reaction product may contain at least 0.5%, about 0.5%, less than 7%, 0.5% to 7%, 5% to 7%, or about 5% lactone byproducts. “Lactone byproducts” or “lactones” as used herein include the one or more lactone byproducts (e.g., L1, L2, L3, and/or L4) present in the reaction product. Additionally or alternatively, the reaction product may contain less than 10%, 5% to 10%, or about 5% 2-furoic acid.

In certain aspects, the resulting FDCA may be isolated and/or purified from the reaction product. For example, the resulting FDCA may be purified and/or isolated by recrystallization techniques or solid/liquid separation. In some aspects, the isolated and/or purified FDCA still includes small amounts of byproducts. The purified product may contain at least 0.1% (1000 ppm) lactone byproducts. In some aspects, the purified product contains less than 0.5% (5000 ppm), or preferably less than 0.25% (2500 ppm) lactone byproducts. In some aspects, the isolated and/or purified FDCA product may contain between about 0.1% to 0.5% lactone byproducts, or between about 0.1% to 0.25% lactone byproducts.

### Synthesis of FDCA using a Halogen Catalyst

In an aspect, FDCA is synthesized from DDG by combining DDG with a solvent and a halogen catalyst. The DDG undergoes a dehydration reaction, removing two water groups. For example, DDG dipotassium salt may be dehydrated to form FDCA:

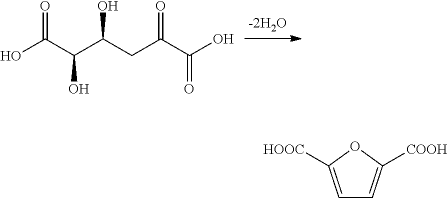 [SMILES: O=C(O)C(=O)C[C@H](O)[C@@H](O)C(=O)O ; O=C(O)c1ccc(C(=O)O)o1]

The catalyst may be a halide (e.g., a halide ion, which may be combined with cations in salts or with protons in acid) or a halogen (e.g., a halogen in its elemental form). In some aspects, the catalyst may be a hydrohalic acid, an alkali or alkaline earth metal salt, a transition metal salt, a rare earth metal salt, a salt in which at least some of the negative ions are halides (e.g., ammonium salts, ionic liquids, ion exchange resins which are exchanged with halides, or salts of other metals), or elemental halogens. When a halide salt includes cations in combination with a halide, the cations may be selected from quaternary ammonium ions, tertiary ammonium ions, secondary ammonium ions, primary ammonium ions, phosphonium ions, or any combination thereof. Elemental halogens may be reduced in situ into halide ions. The catalyst may contain one or more of bromine, chlorine, fluorine, and iodine. For example, a halogen catalyst may be selected from hydrobromic acid, hydrochloric acid, hydrofluoroic acid, hydroiodic acid, sodium bromide, potassium bromide, lithium bromide, rubidium bromide, caesium bromide, magnesium bromide, calcium bromide, strontium bromide, barium bromide, sodium chloride, potassium chloride, lithium chloride, rubidium chloride, caesium chloride, magnesium chloride, calcium chloride, strontium chloride, barium chloride, sodium fluoride, potassium fluoride, lithium fluoride, rubidium fluoride, caesium fluoride, magnesium fluoride, calcium fluoride, strontium fluoride, barium fluoride, sodium iodide, potassium iodide, lithium iodide, rubidium iodide, caesium iodide, magnesium iodide, calcium iodide, strontium iodide, barium iodide, elemental bromine, elemental chlorine, elemental fluorine, elemental iodine, FeBr₃, AlBr₃, NH₄BR, [EMIM]Br, FeCl₃, AlCl₃, NH₄Cl, [EMIM]Clr, FeF₃, AlF₃, NH₄F, [EMIM]F, FeI₃, AlI₃, NH₄I, [EMIM]I, or any combination thereof. In certain aspects, the catalyst includes a hydrohalic acid and a halide salt.

In certain aspects, the hydrohalic acids or halide salts may be used as a solvent in the reaction mixture. In other aspects, the hydrohalic acids or halide salts may form liquid mixtures with DDG at room temperature. Additionally or alternatively, in some aspects, DDG may be treated with gaseous hydrohalic acids. In some aspects, DDG and the halide compound are combined with other solvent(s). In preferred aspects, a halide salt is combined with an acid, such as a hydrohalic acid. By using both a halide salt and a hydrohalic acid the reaction may be catalyzed both with acid and with the beneficial effect of the halide ions. In certain preferred aspects, a catalyst and a solvent are the same compound. For example, a catalyst and a solvent may both be hydrobromic acid, may both be a hydrochloric acid, may both be hydroiodic acid, or may both be hydrofluoric acid.

A solvent that may be combined with a halogen catalyst may be selected from water, acetic acid, propionic acid, butyric acid, trifluoroacetic acid, methanesulfonic acid, sulfuric acid, methanol, ethanol, 1-propanol, 2-propanol, 1-butanol, formic acid, N-methylpyrrolidone, other ionic liquids, or any combination thereof. Various combinations of solvents may include water and acetic acid, water and proprionic acid, and water and trifluoroacetic acid.

The reagents (e.g., DDG, catalyst, solvent) may be combined together in any suitable reaction vessel such as a batch or a continuous reactor. A continuous reactor may be a plug flow reactor, continuous stirred tank reactor, and a continuous stirred tank reactor in series. A reactor may be selected based on its metallurgy. For example, a reactor may be a zirconium reactor, a teflon reactor, a glass-lined reactor, or the like. A preferred reactor may be selected based upon corrosion and chemical compatibility with the halogen being utilized in the dehydration reaction. In some aspects, the reaction vessel is preheated (e.g., preheated to a temperature of 60° C.) prior to initiating a dehydration reaction.

In some aspects, DDG is dissolved in water and then combined with a halogen containing catalyst to form a reaction mixture. The reaction of the reaction mixture may proceed at a temperature within a range of 0° C. to 200° C., alternatively within a range of 30° C. to 150° C., or preferably within a range of 60° C. to 100° C. The pressure in the reaction vessel may be auto generated by the reaction components at the reaction temperature. In some aspects, hydrobromic acid may be combined with water in the reaction vessel and the pressure in the reaction vessel may range from 1 bar to 50 bar. In some aspects, the reaction may proceed (i.e., reach 95% completion) for up to two days if the reaction temperature is low, or the reaction may proceed for less than five minutes if the temperature is 100° C. or higher. A preferred reaction time for the reaction mixture is within the range of one minute to four hours. The reaction may proceed to yield a reaction product including FDCA, water, and other byproducts (e.g., lactones). The FDCA may be filtered and removed from the reaction product.

In some aspects, the reaction may proceed at a fixed temperature. In alternative aspects, the temperature of the reaction mixture may be increased rapidly after the reaction mixture is formed. For example, the temperature of the reaction mixture may be increased from an ambient temperature or from no more than 30° C. to 60° C. or to at least 60° C. within two minutes, alternatively within 5 minutes, or within 20 minutes. In another example, the temperature of the reaction mixture may be increased from an ambient temperature or from no more than 30° C. to 100° C. or to at least 100° C. within two minutes, alternatively within 5 minutes, or within 20 minutes. A fast heat up time, as compared to a slow or gradual temperature increase, can limit and/or prevent side reactions from occurring during the reaction process. By reducing the number of side reactions that occur during the reaction process, the number of byproducts produced during the reaction is reduced. In certain aspects, any byproducts produced by the dehydration reaction are present at below 15%, alternatively less than 12%, alternatively 10% to 12%, or preferably less than 10%.

In some aspects, the halogen catalyst may be added to the reaction mixture in high concentrations. For example, the halogen catalyst added to the reaction mixture may have a halide concentration of greater than 1% by weight, greater than 45% by weight, between 45% to 70% by weight, greater than 55% by weight, between 55% to 70% by weight, or at least 65% by weight of the reaction mixture (including the halide). In some aspects, the halide concentration is 50% by weight, and in other aspects the halide concentration is 62% by weight, with a preferred halide concentration of around 58% by weight of the reaction mixture, including the halide. If both a halide salt and a hydrohalic acid are added to a reaction, the combined halide concentration may be within the range of 55% to 70% by weight of the reaction mixture, including the halide salt and hydrohalic acid.

In preferred aspects, the halogen catalyst and/or solvent contains bromine. In some aspects, the catalyst is selected from a bromide salt, a hydrobromic acid, an elemental bromine ion, or any combination thereof. In certain aspects, the catalyst is hydrobromic acid. Alternatively, the catalyst includes hydrobromic acid and bromide salt. A reaction mixture may contain 1 M to 13 M hydrobromic acid, or in some aspects 2 M to 6 M hydrobromic acid. For example, a reaction mixture may include 40% to 70% water, or alternatively about 38% water, and 10 M to 15 M hydrobromic acid, or alternatively about 12 M hydrobromic acid. The reaction mixture including water and hydrobromic acid may produce a reaction product including FDCA, water and byproducts. The reaction product may include up to 15% byproducts, and 70% to 95% molar yield FDCA.

In other examples, a reaction mixture may include 0% to 30% water, or alternatively about 8% water, 40% to 67% acetic acid, and 1 M to 6 M hydrobromic acid, or alternatively about 5 M hydrobromic acid. The reaction mixture including water, acetic acid, and hydrobromic acid may produce a reaction product including FDCA, water and byproducts. The reaction product may include up to 15% byproducts, and 70% to 95% molar yield FDCA.

Exemplary solvent/catalyst combinations include, but are not limited to, 1) acetic acid, water, and hydrobromic acid; 2) acetic acid and hydrobromic acid; and 3) hydrobromic acid and water. Examples of exemplary process parameters, including a DDG starting material, a solvent, a catalyst, molarity of an acid, molarity of the DDG, reaction time, reaction temperature, molar yield of the FDCA, and any additional comments, such as the volume percent of any water added to the reaction mixture, can be seen in Table 1.

TABLE 1

| | | | [Acid], | [DDG], | | | FDCA | |
|---|---|---|---|---|---|---|---|---|
| Feed | Solvent | Catalyst | M | M | Time, h | Temp, C. | Yield | Comment |
| DDG | Acetic | HBr | 1.0 | | 4 | 60 | 72.89 | |
| 2K | | | | | | | | |
| DDG | Acetic | HBr | 2.9 | | 4 | 60 | 79.05 | |
| 2K | | | | | | | | |
| DDG | Acetic | HBr | 5.14 | 0.10 | 1 | 80 | 91.72 | 8.1% H2O |
| 2K | | | | | | | | by vol. |
| DDG | Acetic | HBr | 5.14 | 0.10 | 2 | 80 | 92.06 | 8.1% H2O |
| 2K | | | | | | | | by vol. |
| DDG | Acetic | HBr | 5.14 | 0.10 | 4 | 80 | 91.90 | 8.1% H2O |
| 2K | | | | | | | | by vol. |
| DDG | Acetic | HBr | 5.14 | 0.10 | 0.0833 | 100 | 87.91 | 8.1% H2O |
| 2K | | | | | | | | by vol. |
| DDG | Acetic | HBr | 5.14 | 0.10 | 0.25 | 100 | 89.79 | 8.1% H2O |
| 2K | | | | | | | | by vol |
| DDG | Acetic | HBr | 5.14 | 0.10 | 0.5 | 100 | 90.44 | 8.1% H2O |
| 2K | | | | | | | | by vol. |
| DDG | Water | HBr | 12.45 | 0.05 | 0.0833 | 100 | 90.24 | 65.78% |
| 2K | | | | | | | | H2O, .05M |
| | | | | | | | | DDG |
| DDG | Water | HBr | 12.45 | 0.05 | 0.25 | 100 | 90.29 | 65.78% |
| 2K | | | | | | | | H2O, .05M |
| | | | | | | | | DDG |
| DDG | Water | HBr | 12.45 | 0.05 | 0.5 | 100 | 90.48 | 65.78% |
| 2K | | | | | | | | H2O, .05M |
| | | | | | | | | DDG |
| DDG | Water | HBr | 12.45 | 0.05 | 1 | 100 | 90.86 | 65.78% |
| 2K | | | | | | | | H2O, .05M |
| | | | | | | | | DDG |
| DDG | Water | HBr | 12.45 | 0.05 | 2 | 100 | 88.90 | 65.78% |
| 2K | | | | | | | | H2O, .05M |
| | | | | | | | | DDG |
| DDG | Water | HBr | 12.45 | 0.05 | 4 | 100 | 87.58 | 65.78% |
| 2K | | | | | | | | H2O, .05M |
| | | | | | | | | DDG |

In other aspects, the halogen catalyst and/or solvent contains chlorine, fluorine, and/or iodine. In some aspects, the catalyst is selected from a halide salt, a hydrohalic acid, an elemental halogen ion, or any combination thereof. In certain aspects, the catalyst is hydrochloric acid. Alternatively, the catalyst includes hydrohalic acid and halide salt. A reaction mixture may contain 1 M to 12 M hydrochloric acid. For example, a reaction mixture may include 63% to 97% water, or alternatively about 70% water, and 1 M to 12 M hydrochloric acid, or alternatively about 11 M hydrochloric acid. The reaction mixture may also contain acetic acid. The reaction mixture including water and hydrochloric acid may produce a reaction product including FDCA, byproducts, and water. The reaction product may include up to 15% byproducts, and 30% to 60% molar yield FDCA.

In other aspects, the catalyst is hydroiodic acid. A reaction mixture may contain 1 M to 8 M hydroiodic acid. In some examples, a reaction mixture may include 40% to 97% water, or alternatively about 50% water, and 3 M to 8 M hydroiodic acid, or alternatively about 7 M hydroiodic acid. The reaction mixture may also contain acetic acid. The reaction mixture including water and hydroiodic acid may produce a reaction product including FDCA, water and byproducts. The reaction product may include up to 15% byproducts, and 30% to 60% molar yield FDCA.

Exemplary solvent/catalyst combinations include, hut are not limited to, 1) acetic acid and hydrochloric acid, 2) water and hydrochloric acid, 3) acetic acid, water, and hydroiodic acid, and 4) water and hydroiodic acid. Examples of exemplary process parameters, including a DDG starting material, a solvent, a catalyst, molarity of an acid, molarity of the DDG, reaction time, reaction temperature, molar yield of the FDCA, and any additional comments, such as the volume percent of any water added to the reaction mixture, can be seen in Table 2.

TABLE 2

| | | | [Acid], | [DDG], | | | FDCA | |
|---|---|---|---|---|---|---|---|---|
| Feed | Solvent | Catalyst | M | M | Time, h | Temp, C. | Yield | Comments |
| DDG | Acetic | HCl | 1.0 | 0.1 | 4 | 100 | 31.0606 | |
| 2K | | | | | | | | |
| DDG | Water | HCl | 11.47 | 0.05 | 4 | 60 | 54.60 | |
| 2K | | | | | | | | |
| DDG | Water | HCl | 11.47 | 0.05 | 4 | 100 | 57.92 | |
| 2K | | | | | | | | |
| DDG | Water | HCl | 11.47 | 0.05 | 1 | 100 | 57.50 | |
| 2K | | | | | | | | |
| DDG | Acetic | HI | 3.0 | 0.1 | 4 | 100 | 33.22 | 29% H2O |
| 2K | | | | | | | | |
| DDG | Acetic | HI | 3.0 | 0.1 | 4 | 100 | 34.23 | 29% H2O |
| DBE | | | | | | | | |
| DDG | Water | HI | 7.20 | 0.05 | 4 | 60 | 41.11 | |
| 2K | | | | | | | | |
| DDG | Water | HI | 6.57 | 0.05 | 4 | 60 | 41.25 | |
| 2K | | | | | | | | |

Although not wishing to be bound by any particular theory, it is possible that the halogen displaces hydroxyl groups of the DDG, thereby aiding in the required dehydration and/or elimination reaction of the DDG due to its enhanced nucleophilicity. Alternatively, it is possible that the halogen may initiate additional dehydration mechanisms that involve the halogen oxidation states. In any event, it was discovered that the yield of FDCA increases if a halogen catalyst is used with the dehydration reaction of DDG to form FDCA.

### Synthesis of FDCA using an Acidic Solvent and Water

In an embodiment of the invention, FDCA is synthesized by combining DDG with water and an acidic solvent and/or catalyst. In some aspects, the water may be used as the principal solvent for the reaction. In other aspects, the water may be added to other solvents, such as acetic acid, to enhance the reaction. In some aspects, an acidic solvent acts as a catalyst (e.g., hydrobromic acid). An acidic solvent may be selected from hydrochloric acid, hydroiodic acid, hydrobromic acid, hydrofluoric acid, acetic acid, sulfuric acid, phosphoric acid, nitric acid, trifluoroacetic acid, methanesulfonic acid, ethanesulfonic acid, benzenesulfonic acid, p-toluenesulfonic acid, acidic ion exchange resins, other supported sulfonic acids (which may include, e.g., Nafion, Amberlyst®-15, other sulfonic acid resins, and the like), other heterogeneous acid catalysts, heteropoly acids (which may include, e.g., tungstosilicic acid, phosphomolybdic acid, phosphotungstic acid, and the like), acids with a first pKa of less than 2, other supported organic, inorganic, and supported or solid acids, and combinations thereof.

In certain, aspects, DDG is combined with water and an acidic solvent to form a reaction mixture. In some aspects, a catalyst is added to the reaction mixture. The catalyst may be selected from a halide salt (e.g., alkali metal halides, alkaline earth metal halides, transition metal halides, rare earth metal halides, or organic cations (e.g., quaternary ammonium ions, tertiary ammonium ions, secondary ammonium ions, primary ammonium ions, or phosphonium ions) in combination with halide ions), a hydrohalic acid, an elemental ion, and any combination thereof. The catalyst may be selected from sodium chloride, potassium chloride, lithium chloride, rubidium chloride, caesium chloride, magnesium chloride, calcium chloride, strontium chloride, barium chloride, FeCl₃, AlCl₃, NH₄Cl, [EMIM]Cl, sodium fluoride, potassium fluoride, lithium fluoride, rubidium fluoride, caesium fluoride, magnesium fluoride, calcium fluoride, strontium fluoride, barium fluoride, FeF₃, AlF₃, NH₄F, [EMIM]F, sodium iodide, potassium iodide, lithium iodide, rubidium iodide, caesium iodide, magnesium iodide, calcium iodide, strontium iodide, barium iodide, FeI₃, AlI₃, NH₄I, [EMIM]I, sodium bromide, potassium bromide, lithium bromide, rubidium bromide, caesium bromide, magnesium bromide, calcium bromide, strontium bromide, barium bromide, FeBr₃, AlBr₃, NH₄Br, [EMIM]Br, and combinations thereof.

The reagents (e.g., DDG, water, acidic solvent) may be combined together in any suitable reaction vessel such as a batch or a continuous reactor. A continuous reactor may be a plug flow reactor, continuous stirred tank reactor, and a continuous stirred tank reactor in series. A reactor may be selected based on its metallurgy. For example, a reactor may be a zirconium reactor, a teflon reactor, a glass-lined reactor, or the like. A preferred reactor may be selected based upon corrosion and chemical compatibility with the reaction mixture of the dehydration reaction. In some aspects, the reaction vessel is preheated (e.g., preheated to a temperature of 60° C.) prior to initiating a dehydration reaction.

In some aspects, DDG is dissolved in water and then combined with an acidic solvent and an additional volume of water. The reaction of the reaction mixture may proceed at a temperature within a range of 0° C. to 200° C., alternatively within a range of 30° C. to 150° C., or preferably within a range of 60° C. to 100° C. The pressure in the reaction vessel may be auto generated by the reaction components at the reaction temperature. The pressure in the reaction vessel may range from 1 bar to 17 bar. In some aspects, the reaction may proceed (i.e., achieve 95% completion) for up to two days if the reaction temperature is low, or the reaction may proceed for less than five minutes if the temperature is 100° C. or higher. A preferred reaction time for the reaction mixture is within the range of one minute to four hours. The reaction may proceed to yield a reaction product including FDCA, water, and other byproducts (e.g., lactones). The FDCA may be filtered and removed from the reaction product.

In some aspects, the reaction may proceed at a fixed temperature. In alternative aspects, the temperature of the reaction mixture may be increased rapidly after the reaction mixture is formed. For example, the temperature of the reaction mixture may be increased from an ambient temperature or from no more than 30° C. to 60° C. or at least 60° C. within two minutes, alternatively within 5 minutes, or within 20 minutes. In another example, the temperature of the reaction mixture may be increased from an ambient temperature or from no more than 30° C. to 100° C. or to at least 100° C. within two minutes, alternatively within 5minutes, or within 20 minutes. A fast heat up time, as compared to a slow or gradual temperature increase, can limit and/or prevent side reactions from occurring during the reaction process. By reducing the number of side reactions that occur during the reaction process, the number of byproducts produced during the reaction is reduced. In certain aspects, any byproducts produced by the dehydration reaction are present at below 15%, alternatively less than 12%, alternatively 10% to 12%, or preferably less than 10%.

In some aspects, water may be added to the reaction mixture. The including of water can have a significant impact on the reaction and yield. For example, water can be in the reaction mixture in an amount (by volume) of at least 10%, at least 20%, at least 30%, 10% to 70%, 10% to 30%, or 30% to 65%. In preferred embodiments, the reaction mixture includes water and hydrobromic acid. The reaction mixture may contain 1 M to 13 M hydrobromic acid, or in some aspects 2 M to 6 M hydrobromic acid. For example, a reaction mixture may include 10% to 70% water, or alternatively 30% to 65% water, and 10 M to 15 M hydrobromic acid, or alternatively about 12 M hydrobromic acid. The reaction mixture including water and hydrobromic acid may produce a reaction product including FDCA, byproducts, and water. The reaction product may include up to 15% byproducts, and 40% to 95% molar yield FDCA.

Exemplary solvent/catalyst combinations include, but are not limited to, 1) water and hydrobromic acid; 2) water and hydrochloric acid; 3) water and hydroiodic acid; 4) water and methanesulfonic acid; and 5) water, acetic acid and sulfuric acid. Examples of exemplary process parameters, including a DDG starling material, a solvent, a catalyst, molarity of an acid, molarity of the DDG, reaction time, reaction temperature, molar yield of the FDCA, and any additional comments, such as the volume percent of any water added to the reaction mixture, can be seen in Table 3.

TABLE 3

| | | | [Acid], | [DDG], | | | FDCA | |
|---|---|---|---|---|---|---|---|---|
| Feed | Solvent | Catalyst | M | M | Time, h | Temp, C. | Yield | Comments |
| DDG | Water | HBr | 12.45 | 0.05 | 0.0833 | 100 | 90.24 | 65.78% |
| 2K | | | | | | | | H2O, .05M |
| | | | | | | | | DDG |
| DDG | Water | HBr | 12.45 | 0.05 | 0.25 | 100 | 90.29 | 65.78% |
| 2K | | | | | | | | H2O, .05M |
| | | | | | | | | DDG |
| DDG | Water | HBr | 12.45 | 0.05 | 0.5 | 100 | 90.48 | 65,78% |
| 2K | | | | | | | | H2O, .05M |
| | | | | | | | | DDG |
| DDG | Water | HBr | 12.45 | 0.05 | 1 | 100 | 90.86 | 65.78% |
| 2K | | | | | | | | H2O .05M |
| | | | | | | | | DDG |
| DDG | Water | HBr | 12.45 | 0.05 | 2 | 100 | 88.90 | 65.78% |
| 2K | | | | | | | | H2O, .05M |
| | | | | | | | | DDG |
| DDG | Water | HBr | 12.45 | 0.05 | 4 | 100 | 87.58 | 65.78% |
| 2K | | | | | | | | H2O, .05M |
| | | | | | | | | DDG |
| DDG | Water | HCl | 11.47 | 0.05 | 4 | 60 | 54.60 | |
| 2K | | | | | | | | |
| DDG | Water | HCl | 11.47 | 0.05 | 4 | 100 | 57.92 | |
| 2K | | | | | | | | |
| DDG | Water | HCl | 11.47 | 0.05 | 1 | 100 | 57.50 | |
| 2K | | | | | | | | |
| DDG | Water | HI | 7.20 | 0.05 | 4 | 60 | 41.11 | |
| 2K | | | | | | | | |
| DDG | Water | HI | 6.57 | 0.05 | 4 | 60 | 41.25 | |
| 2K | | | | | | | | |
| DDG | MSA | MSA | 13.9 | | 4 | 100 | 43.88 | 10% H2O |
| 2K | | | | | | | | |
| DDG | Acetic | H2SO4 | 5.1 | | 4 | 100 | 34.19 | 10% H2O |
| 2K | | | | | | | | |

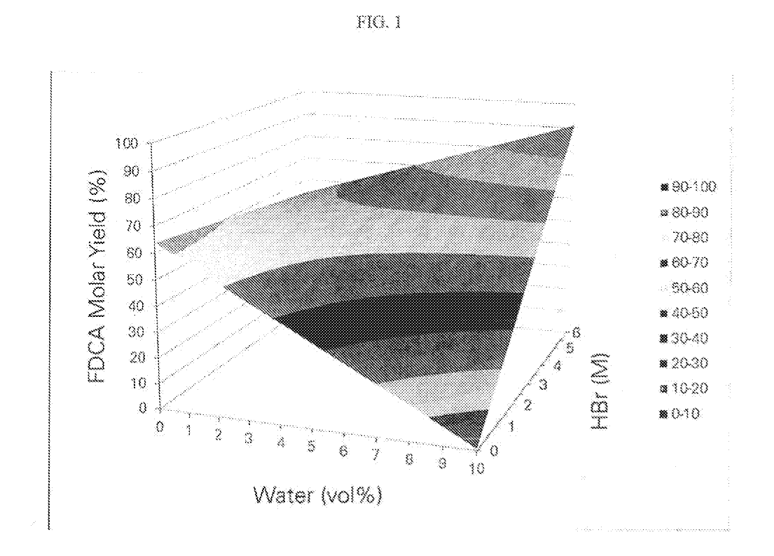

Conditions for various alternative dehydration reactions utilizing DDG-2K as the starting material are provided in Table 4. The first line for each acid provides a working range for each reaction condition and the subsequent line(s) provides examples of specific reaction conditions. As seen in FIG. 1, higher molar yields of FDCA may be obtained when utilizing both water and hydrobromic acid in dehydration reactions.

TABLE 4

| | | | | | Highest |
|---|---|---|---|---|---|
| | | | | | FDCA |
| | Concentration | Water | Temp. | Time | Yield |
| Acid | (M) | (vol %) | (° C.) | (h) | (%) |
| H₂SO₄ | 0.25-18 | 0-30 | 60-160 | 2-4 | |
| | 9.0 | 0 | 60 | 4 | 40 |
| | 5.1 | 10 | 100 | 4 | 34 |
| H₃PO₄ | 2.1-5.1 | 10-30 | 60-100 | 2-4 | |
| | 5.1-10 | 10 | 100 | 4 | 2 |
| Methanesulfonic | 1.0-13.9 | 5-10 | 60-100 | 4 | |
| acid | | | | | |
| | 13.9 | 10 | 60 | 4 | 44 |
| p-Toluenesulfonic | 1.0-3.0 | 7-10 | 100 | 4 | |
| acid | | | | | |
| | 3.0 | 10 | 100 | 4 | 17 |
| Amberlyst-15 | 1.57 eq | 10 | 100 | 4 | 15 |
| H₄SiW₁₂O₄₀ | 0.2 | 5 | 100 | 4 | 14 |
| H₃PMo₁₂O₄₀ | 0.2 | 5 | 100 | 4 | 5 |
| H₃PW₁₂O₄₀ | 0.2 | 5 | 100 | 4 | 6 |
| HCl | 1.0 | 0 | 60-100 | 4 | |
| | 1.0 | 0 | 100 | 4 | 31 |
| HBr | 0.5-5.1 | 0-30 | 60-160 | 0.5-24 | |
| | 5.1 | 9 | 60 | 4 | 93 |
| | 1.0 | 0 | 60 | 4 | 73 |
| | 5.1 | 10 | 100 | 4 | 86 |
| | 2.1 | 30 | 100 | 4 | 39 |
| HI | 1.6-3.0 | 0-29 | 60-100 | 4 | |
| | 3.0 | 29 | 100 | 4 | 34 |
| | 3.0 | 29 | 60 | 4 | 23 |

It was unexpected that the addition of water to the reaction mixture would increase the yield of a product in a dehydration reaction because water is the product of dehydration, and by Le Chateliers' principle increased concentrations of water would be expected to disfavor dehydration chemistry. Although not wishing to be bound by any particular theory, possible reasons for the advantageous effect of water may be good solubility of DDG and acids in water, low solubility of FDCA in water, stabilization of transition states for dehydration chemistry by the polar solvent, and the preference of DDG for furanoid forms in water, which are pre-disposed for dehydration into FDCA.

Additionally, water may be an advantageous solvent for the dehydration of DDG to FDCA because the water causes the DDG to assume a furanoid form that is better for dehydration reactions. The furanoid forms of DDG are 5-membered rings which may be easy to dehydrate into FDCA. When the DDG assumes its preferred form it produces fewer byproducts during the dehydration reaction, as well as encouraging a more efficient (e.g., faster) reaction.

FDCA may be further isolated at a high purity (e.g., about 99%) from the above described reactions by filtrating and washing the FDCA product with water only.

### Synthesis of FDCA using a Carboxylic Acid

In an embodiment of the invention, FDCA is synthesized from DDG in combination with a carboxylic acid. For example, DDG may be dehydrated to form FDCA in a carboxylic acid solvent:

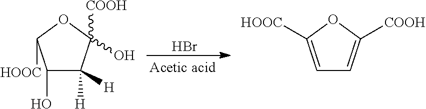

A carboxylic acid may be combined with DDG to produce a reaction product including FDCA. In some aspects, the carboxylic acid and DDG are combined with a solvent and/or a catalyst. In other aspects, the carboxylic acid acts as both a solvent and a catalyst. For example, a carboxylic acid with a low pKa (e.g., less than 3.5) may act as both a solvent and a catalyst in the reaction. In some aspects, a catalyst may be added to the carboxylic acid having a low pKa to speed up the reaction of DDG to FDCA. In another example, a carboxylic acid with a high pKa (e.g., greater than 3.5) may be combined with a catalyst, and in some aspects a solvent. In some aspects, a carboxylic acid may be selected from trifluoroacetic acid, acetic acid, acetic acid, propionic acid, butyric acid, other carboxylic acids with a low pKa (e.g., less than 3.5 or a pKa less than 2.0), other carboxylic acids with a high pKa (e.g., greater than 3.5), and any combination thereof.

In some aspects, a solvent is added to the reaction mixture in addition to the carboxylic acid. Solvents may be selected from water, methanol, ethanol, 1-propanol, 2-propanol, 1-butanol, N-methylpyrrolidone, other ionic liquids, or any combination thereof. In certain aspects, the dehydration reaction may utilize three solvents in combination. In alternative aspects, the dehydration reaction may utilize two solvents in combination. In still other aspects, the dehydration reaction may utilize a single solvent.

In certain aspects, a catalyst is added to the reaction mixture. The catalyst may be selected from a halide salt (e.g., alkali metal halides, alkaline earth metal halides, transition metal halides, rare earth metal halides, or organic cations (e.g., quaternary ammonium ions, tertiary ammonium ions, secondary ammonium ions, primary ammonium ions, or phosphonium ions) in combination with halide ions), a hydrohalic acid, elemental ions, a strong acid, or any combination thereof. For example, the catalyst may be selected from sodium chloride, potassium chloride, lithium chloride, rubidium chloride, caesium chloride, magnesium chloride, calcium chloride, strontium chloride, barium chloride, FeCl₃, AlCl₃, NH₄Cl, [EMIM], sodium fluoride, potassium fluoride, lithium fluoride, rubidium fluoride, caesium fluoride, magnesium fluoride, calcium fluoride, strontium fluoride, barium fluoride, FeF₃, AlF₃, NH₄F, [EMIM]F, sodium iodide, potassium iodide, lithium iodide, rubidium iodide, caesium iodide, magnesium iodide, calcium iodide, strontium iodide, barium iodide, FeI₃, AlI₃, NH₄I, [EMIM]I, sodium bromide, potassium bromide, lithium bromide, rubidium bromide, caesium bromide, magnesium bromide, calcium bromide, strontium bromide, barium bromide, FeBr₃, AlBr₃, NH₄Br, [EMIM]Br, hydrobromic acid, hydroiodic acid, hydrofluoric acid, hydrochloric acid, elemental bromine, elemental chlorine, elemental fluorine, elemental iodine, methanesulfonic acid, trifluoromethanesulfonic acid, sulfuric acid, and combinations thereof.

The reagents (e.g., DDG, catalyst, solvent) may be combined together in any suitable reaction vessel such as a batch or a continuous reactor. A continuous reactor may be a plug flow reactor, continuous stirred tank reactor, and a continuous stirred tank reactor in series. A reactor may be selected based on its metallurgy. For example, a reactor may be a zirconium reactor, a teflon reactor, glass-lined reactor or the like. A preferred reactor may be selected based upon corrosion and chemical compatibility with the carboxylic acid being utilized in the dehydration reaction. In some aspects, the reaction vessel is preheated (e.g., preheated to a temperature of 60° C.) prior to initiating a dehydration reaction.

In some aspects, DDG is dissolved in water and then combined with a carboxylic acid, and in some instances a catalyst and/or solvent, to form a reaction mixture. The reaction of the reaction mixture may proceed at a temperature within a range of 0° C. to 200° C., alternatively within a range of 30° C. to 150° C., or preferably within a range of 60° C. to 100° C. The pressure in the reaction vessel may be auto generated by the reaction components at the reaction temperature. In some aspects, acetic acid may be used in the reaction vessel and the pressure in the reaction vessel may range from 1 bar to 10 bar. In some aspects, the reaction may proceed for up to two days if the reaction temperature is low, or the reaction may proceed for less than five minutes if the temperature is 100° C. or higher. A preferred reaction time (i.e., time to achieve 95% completion) for the reaction mixture is within the range of one minute to four hours. The reaction may proceed to yield a reaction product including FDCA, water, and other byproducts (e.g., lactones). The FDCA may be filtered and removed from the reaction product.

In some aspects, the reaction may proceed at a fixed temperature. In alternative aspects, the temperature of the reaction mixture may be increased rapidly after the reaction mixture is formed. For example, the temperature of the reaction mixture may be increased front an ambient temperature or from no more than 30° C. to 60° C. or to at least 60° C. within two minutes, alternatively within 5 minutes, or within 20 minutes. In another example, the temperature of the reaction mixture may be increased from an ambient temperature or from no more than 30° C. to 100° C. or to at least 100° C. within two minutes, alternatively within 5 minutes, or within 20 minutes. A fast heat up time, as compared to a slow or gradual temperature increase, can limit and/or prevent side reactions from occurring during the reaction process. By reducing the number of side reactions that occur during the reaction process, the number of byproducts produced during the reaction is reduced. In certain aspects, any byproducts produced by the dehydration reaction are present at below 15%, alternatively less than 12%, alternatively 10% to 12%, or preferably less than 10%.

In preferred aspects, the carboxylic acid is trifluoroacetic acid. A reaction mixture may contain trifluoroacetic acid and hydrobromic acid. For example, a reaction mixture may include 0 M to 6.0 M hydrobromic acid, or alternatively about 3 M hydrobromic acid. The reaction mixture including hydrobromic acid and trifluoroacetic acid may produce a reaction product including FDCA, byproducts, and water. The reaction product may include up to 15% byproducts, and 50% to 80% molar yield FDCA. In some additional examples, water may be added to the reaction mixture. In certain aspects, 5 vol % to 30 vol % of the reaction mixture is water.

Exemplary catalyst or catalyst/solvent combinations include, but are not limited to, 1) trifluoroacetic acid and sulfuric acid; 2) acetic acid and hydrobromic acid; 3) hydrobromic acid, trifluoroacetic acid, and water; and 4) hydrobromic acid, trifluoroacetic acid, acetic acid, and water. Examples of exemplary process parameters, including a DDG starting material, a solvent, a catalyst, molarity of an acid, molarity of the DDG, reaction time, reaction temperature, molar yield of the FDCA, and any additional comments, such as the volume percent of any water added to the reaction mixture, can be seen in Table 5.

TABLE 5

| | | | [Acid], | [DDG], | | | FDCA | |
|---|---|---|---|---|---|---|---|---|
| Feed | Solvent | Catalyst | M | M | Time, h | Temp, C. | Yield | Comments |
| DDG | TFA | H2SO4 | 0.9 | | 4 | 60 | 17.35 | |
| 2K | | | | | | | | |
| DDG | Acetic | HBr | 1.0 | | 4 | 60 | 72.89 | |
| 2K | | | | | | | | |
| DDG | Acetic | HBr | 2.9 | | 4 | 60 | 79.05 | |
| 2K | | | | | | | | |
| DDG | TFA | HBr | 0.6 | | 4 | 100 | 56.43 | 10% H2O |
| 2K | | | | | | | | |
| DDG | TFA | HBr | 3.1 | | 4 | 100 | 60.94 | 30% H2O |
| 2K | | | | | | | | |
| DDG | TFA/Acetic | HBr | 5.1 | | 4 | 60 | 75.11 | 30% H2O |
| 2K | | | | | | | | |
| DDG | TFA/Acetic | HBr | 5.1 | | 4 | 100 | 70.45 | 30% H2O |
| 2K | | | | | | | | |

Conditions for various alternative dehydration reactions utilizing DDG-2K as the starting material in combination with trifluoroacetic acid, acetic acid, or trifluoroacetic acid and acetic acid in combination are provided in Table 6.

TABLE 6

| | | Water | Temp | | Molar Yield |
|---|---|---|---|---|---|
| Solvent | Acid (M) | (vol %) | (° C.) | Time (h) | of FDCA (%) |
| TFA | | 0 | 60 | 4 | 1 |
| TFA | | 5 | 60 | 4 | 0 |
| TFA | H₂SO₄ (0.9) | 0 | 60 | 4 | 17 |
| TFA | H₂SO₄ (0.9) | 5 | 60 | 4 | 4 |
| TFA | HBr (0.6) | 10 | 60 | 4 | 14 |
| TFA | HBr (0.6) | 10 | 60 | 4 | 56 |
| TFA | HBr (3.1) | 30 | 100 | 4 | 61 |
| TFA/Acetic | HBr (5.1) | 30 | 100 | 4 | 70 |
| Acetic | HBr (2.1) | 30 | 100 | 4 | 39 |
| Acetic | HBr (5.1) | 30 | 100 | 4 | 73 |
| TFA | LiBr (2.1) - | 10 | 100 | 4 | 49 |
| | no added | | | | |
| | strong acid | | | | |

It was unexpected for carboxylic acids to act as an effective medium for the dehydration reaction of DDG to FDCA. Although not wishing to be bound by any particular theory, carboxylic acids may be an advantageous solvent and/or catalyst for the dehydration of DDG to FDCA because the carboxylic acid causes the DDG to assume furanoid forms that are better tor dehydration reactions. The furanoid forms of DDG are 5-membered rings which may be easy to dehydrate into FDCA. When the DDG assumes its preferred form it produces fewer byproducts during the dehydration reaction, as well as encouraging a more efficient (e.g., fester) reaction.

Acetic acid may be an advantageous solvent for the dehydration of DDG to FDCA because DDG and other acids have good solubility in acetic acid, FDCA has low solubility in acetic acid, transition states for dehydration chemistry are stabilized by the polar solvent, and DDG prefers furanoid forms in acetic acid, which are predisposed for dehydration into FDCA. Other carboxylic acids exhibit similar characteristics. Additionally, it is believed that carboxylic acid solvents enhance the acidity of other acids (e.g., hydrobromic acid, hydrochloric acid, and the like) which are used as acid catalysts in combination with these solvents. Further, carboxylic acids having a low pKa (e.g., less than 3.5), such as trifluoroacetic acid, form a distinct class within the carboxylic acids. In contrast to acetic acid (pKa of 4.76), these acids have enhanced acidity which is understood as accelerating the dehydration reaction of DDG to FDCA.

### EXAMPLE

It will be appreciated that many changes may be made to the following examples, while still obtaining similar results. Accordingly, the following examples, illustrating embodiments of processing DDG to obtain FDCA utilizing various reaction conditions and reagents, are intended to illustrate and not to limit the invention.

### Example 1

DDG dipotassium salt is combined with 0.25 M H₂SO₄ in acetic acid. The reaction proceeds at 60° C. for 4 hours yielding 1% FDCA molar yield.

### Example 2

DDG dipotassium salt is combined with 0.25 M H₂SO₄ in acetic acid with NaBr (8 wt %). The reaction proceeds at 60° C. for 4 hours yielding 19% FDCA molar yield.

### Example 3

DDG dipotassium salt is combined with 0.25 M H₂SO₄ in acetic acid. The reaction proceeds at 160° C. for 3 hours to produce 20% FDCA molar yield.

### Example 4

DDG dipotassium salt is combined with 0.25 M H₂SO₄ in acetic acid with NaBr (0.7 wt %). The reaction proceeds at 160° C. for 3 hours to produce 31% FDCA molar yield.

### Example 5

DDG dibutyl ester is combined with 9 M H₂SO₄ in 1-butanol. The reaction proceeds at 60° C. for 2 hours yielding 53% FDCA molar yield.

### Example 6

DDG dibutyl ester is combined with 9 M H₂SO₄ in acetic acid. The reaction proceeds at 60° C. for 1 hour yielding 22% FDCA-DBE molar yield.

### Example 7

DDG dibutyl ester is combined with 1 M HCl in acetic acid. The reaction proceeds at 60° C. for 4 hours yielding 43% FDCA-DBF molar yield.

### Example 8

DDG dibutyl ester is combined with 2.9 M HBr in acetic acid. The reaction proceeds at 60° C. for 4 hours yielding 61% FDCA-DBE molar yield.

### Example 9

0.1 M DDG 2K is combined with 5.7 M HBr in acetic acid. The reaction proceeds at 60° C. for 4 hours yielding 33% FDCA molar yield.

### Example 10

0.1 M DDG 2K is combined with 2.9 M HBr in acetic acid. The reaction proceeds at 60° C. for 4 hours to produce 82% FDCA molar yield.

### Example 11

0.1 M DDG 2K is combined with 5.7 M HBr in acetic acid with 10 vol % water. The reaction proceeds at 60° C. for 4 hours yielding 89% FDCA molar yield.

### Example 12

0.1 M DDG 2K is combined with 5.1 M HBr in acetic acid with 10 vol % water. The reaction proceeds at 60° C. for 4 hours yielding 91% FDCA molar yield.

### Example 13

0.05 M DDG 2K is combined with 12.45 M HBr in water. The reaction proceeds at 100° C. for 1 hour yielding 77% FDCA molar yield.

### Example 14

0.05 M DDG 2K is combined with 5.2 M HBr in acetic acid with 8.2 vol % water. The reaction proceeds at 100° C. for 4 hours yielding 71% FDCA molar yield.

### Example 15

DDG-DBE is combined with 9 M H₂SO₄ in 1-butanol. The reaction proceeds at 60° C. for 2 hours yielding 53% FDCA-DBE molar yield.

### Example 16

DDG-DBE is combined with 2.9 M HBr in acetic acid. The reaction proceeds at 60° C. for 4 hours yielding 52% FDCA-DBE molar yield.

### Example 17

DDG-DBE is combined with 9 M H₂SO₄ in 1-butanol. The reaction proceeds at 60° C. for 2 hours yielding 53% FDCA-DBE molar yield.

### Example 18

DDG-DBE is combined with 2.9 M HBr in acetic acid. The reaction proceeds at 60° C. for 4 hours yielding 52% FDCA-DBE molar yield.

### Example 19

DDG-DBE is combined with trifluoroacetic acid. The reaction proceeds at 60° C. for 4 hours yielding 77% FDCA-DBE molar yield.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, the steps described may be performed in other than the recited order unless stated otherwise, and one or more steps illustrated may be options in accordance with aspects of the disclosure.

1-23. (canceled)

24. A method of producing 2,5-furandicarboxylic acid comprising:
mixing a solution including 4-deoxy-5-dehydroglucaric acid and water with an acidic solvent and a catalyst in a reaction vessel to form a reaction mixture;
heating the reaction mixture to a temperature no greater than 150° C.;
allowing the 4-deoxy-5-dehydroglucaric acid to react in the presence of the water and the acidic solvent to produce 2,5-furandicarboxylic acid, water, and byproducts;
removing the water produced during the reaction continuously or periodically; and
removing the 2,5-furandicarboxlic acid from the reaction product,
wherein the acidic solvent is selected from the group consisting of hydrochloric acid, hydroiodic acid, hydrobromic acid, hydrofluoric acid, acetic acid, sulfuric acid, phosphoric acid, nitric acid, trifluoroacetic acid, methanesulfonic acid, ethanesulfonic acid, benzenesulfonic acid, p-toluenesulfonic acid, acidic ion exchange resins, tungstosilicic acid, phosphomolybdic acid, phosphotungstic acid, and combinations thereof,
wherein the catalyst is selected from the group consisting of sodium chloride, potassium chloride, lithium chloride, rubidium chloride, cesium chloride, magnesium chloride, calcium chloride, strontium chloride, barium chloride, FeCl₃, AlCl₃, NH4Cl, [EMIM]Cl, sodium fluoride, potassium fluoride, lithium fluoride, rubidium fluoride, cesium fluoride, magnesium fluoride, calcium fluoride, strontium fluoride, barium fluoride, FeF₃, AlF₃, NH₄F, [EMIM]F, sodium iodide, potassium iodide, lithium iodide, rubidium iodide, cesium iodide, magnesium iodide, calcium iodide, strontium iodide, barium iodide, FeI₃, AlI₃, NH₄I, [EMIM]I, sodium bromide, potassium bromide, lithium bromide, rubidium bromide, cesium bromide, magnesium bromide, calcium bromide, strontium bromide, barium bromide, FeBr₃, AlBr₃, NH₄Br, [EMIM]Br, and combinations thereof,
wherein the total amount of water in the reaction mixture is at least 10 vol % of the reaction mixture, and
wherein the byproducts produced include lactones.

25. The method of claim 24, wherein the acidic solvent is selected from the group consisting of hydrochloric acid, hydroiodic acid, hydrobromic acid, hydrofluoric acid, and combinations thereof.

26. The method of claim 24, wherein the total amount of water in the reaction mixture is at least 30 vol % of the reaction mixture.

27. The method of claim 24, further comprising preheating the reaction vessel to a temperature of 60° C. before mixing the solution including the 4-deoxy-5-dehydroglucaric acid and water with the solvent and the catalyst in the reaction vessel.

28. The method of claim 24, wherein the 2,5-furandicarboxylic acid has a yield of greater than 40 mol %.

29. A composition of 2,5-furandicarboxylic acid including at least 85 wt % 2,5-furandicarboxylic acid and at least one byproduct selected from one or more of 2-furoic acid and lactones, prepared by a method comprising:
mixing 4-deoxy-5-dehydroglucaric acid with water and an acidic solvent to form a reaction mixture; and
allowing the 4-deoxy-5-dehydroglucaric acid to react in the presence of the water and acidic solvent to produce 2,5-furandicarboxylic acid, water, and byproducts.
